(19) United States
(12) Patent Application Publication
(10) Pub. No.: US 2012/0288927 A1
(43) Pub. Date: Nov. 15, 2012

(54) BIOMASS TREATMENT DEVICE

(76) Inventors: Norimitsu Kaneko, Yokohama-shi (JP); Makoto Kitano, Tokyo (JP); Kenji Sato, Fujisawa-shi (JP); Kentaro Nariai, Yokohama-shi (JP); Michikazu Hara, Yokohama-shi (JP); Daizo Yamaguchi, Tsuyama-shi (JP)

(21) Appl. No.: 13/522,602

(22) PCT Filed: Jan. 18, 2011
(86) PCT No.: PCT/JP2011/050766

§ 371 Date: Jul. 17, 2012

(30) Foreign Application Priority Data
Jan. 18, 2010 (JP) P2010-008558

(51) Int. Cl. B01J 19/00; C12M 1/02

(52) U.S. Cl. 435/289.1; 127/1

(57) ABSTRACT

A biomass treatment device (A) includes a hot compressed water reaction device (1) that hydrolyzes a biomass by passing hot compressed water to the biomass to prepare polysaccharides, and a solid-acid catalytic reaction device (2 and 3) that generates monosaccharides from the polysaccharides using a solid-acid catalyst, and the device includes at least one of a first heat exchanger (1b and 1b′) for heating the hot compressed water by the heat of a monosaccharide solution including the monosaccharides delivered from the solid-acid-catalytic reaction device and a second heat exchanger (1c) for heating the hot compressed water by the heat of a polysaccharide solution including the polysaccharides introduced into the solid-acid-catalytic reaction device from the hot compressed water reaction device. According to the biomass treatment device, it is possible to improve the energy efficiency more than the related art.

### TECHNICAL FIELD

The present invention relates to a biomass treatment device.

This application claims priority to and the benefit of Japanese Patent Application No. 2010-8558 filed on Jan. 18, 2010, the disclosure of which is incorporated by reference herein.

### BACKGROUND ART

In the following Patent Document 1, as an efficient method of saccharifying a biomass, a hot compressed water reaction device for generating polysaccharides (xylooligosaccharide and cellooligosaccharide) by hydrolyzing a biomass feedstock with hot compressed water (240° C. to 340° C.) and a solid-acid-catalytic reaction device for monosaccharifying the polysaccharides at the latter-stage are proposed.

### PRIOR ART DOCUMENT

### Patent Document

[Patent Document 1] Japanese Patent Application, First Publication No. 2009-77697

### SUMMARY OF THE INVENTION

### Problems to be Solved by the Invention

However, in the related art mentioned above, a tremendous amount of energy for heating is necessary since hot water heated to 240° C. to 340° C. should be continuously supplied to the biomass feedstock. In addition, the biomass feedstock is monosaccharified with the solid-acid-catalytic reaction device at the latter-stage after being treated with a hot compressed water reaction device. However, a temperature higher than room temperature should be maintained to facilitate the solid-acid-catalyzed reaction at the latter-stage. Because of this, a tremendous amount of energy is necessary in the solid-acid-analyzed reaction device.

In consideration of the above-mentioned circumstances, it is an object of the present invention to improve energy efficiency in the saccharification treatment of a biomass more than the related art.

### Means for Solving the Problems

In order to accomplish the above object, a biomass treatment device according to the present invention includes a hot compressed water reaction device that hydrolyzes a biomass by passing hot compressed water to the biomass to prepare polysaccharides, and a solid-acid catalytic reaction device that generates monosaccharides from the polysaccharides using the solid-acid catalyst. The biomass treatment device includes at least one of a first heat exchanger for heating the hot compressed water by the heat of a monosaccharide solution including the monosaccharides delivered from the solid-acid-catalytic reaction device and a second heat exchanger for heating the hot compressed water by the heat of a polysaccharide solution including the polysaccharides introduced into the solid-acid-catalytic reaction device from the hot compressed water reaction device.

Further, in the biomass treatment device according to the present invention, when the temperature of the monosaccharide solution is lower than the temperature of the polysaccharide solution, the hot compressed water may be heated by a second heat exchanger after the first heat exchanger.

Further, in the biomass treatment device according to the present invention, more than one solid-acid-catalystic reaction device may be included, and the hot compressed water reaction device may deliver the polysaccharide solutions to the each solid-acid-catalytic reaction device apart according to the type of polysaccharides. Water for the hot compressed water may be branched into multi-streams and each water stream is heated by the first heat exchanger that is installed in each of the solid-acid-catalytic reaction devices.

Further, the biomass treatment device according to the present invention may further include an enzyme reactor for hydrolyzing the remined biomass after the hot compressed water has passed with an enzyme.

In this case, the biomass treatment device may further include a third heat exchanger for heating the solution delivered from the enzyme reactor. The solution heated by the third heat exchanger is delivered to the solid-acid-catalytic reaction devices.

Further, in the third heat exchanger of the present invention, the solution delivered from the enzyme reactor may be heated by the heat of the monosaccharide solution delivered from the solid-acid-catalytic reaction device in the third heat exchanger.

### Effect of the Invention

The biomass treatment device according to the present invention includes at least one of a first heat exchanger for heating the hot compressed water by the heat of a monosaccharide solution including monosaccharides delivered from the solid-acid-catalytic reaction device and a second heat exchanger for heating the hot compressed water by the heat of a polysaccharide solution including polysaccharides introduced into the solid-acid-catalytic reaction device from the hot compressed water reaction device. Thus, since hot compressed water is heated using heat of at least one of the monosaccharide solution and the polysaccharide solution, it is possible to improve the energy efficiency in heating of the hot compressed water more than the related art.

### BRIEF DESCRIPTION OF THE DRAWINGS

FIG. 1 is a functional block diagram showing one example of a biomass treatment device according to an embodiment of the present invention.

FIG. 2 is a functional block diagram showing a modified example of the biomass treatment device according to an embodiment of the present invention.

### MODES FOR CARRYING OUT THE INVENTION

Hereinafter, example embodiments of the present invention will be described in detail with reference to the accompanying drawings.

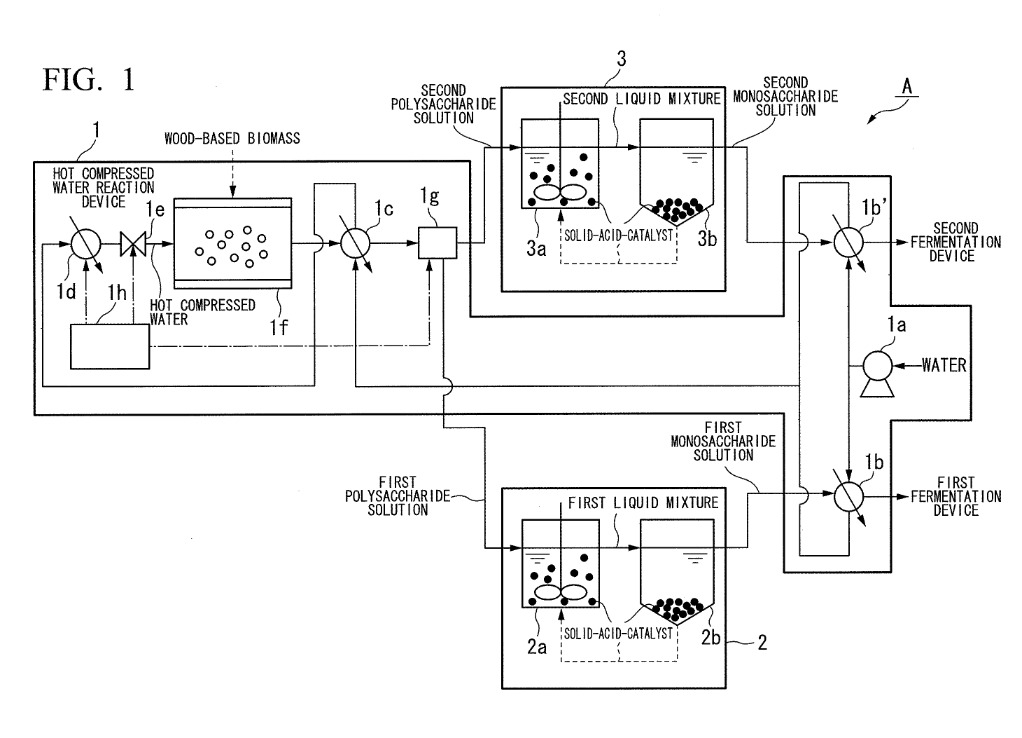

A schematic configuration of a biomass treatment device according to the embodiment is shown in FIG. 1. The biomass treatment device A includes a hot compressed water reaction device 1, a first catalytic reaction device 2, and a second catalytic reaction device 3.

The biomass treatment device A is a device for generating polysaccharides through hydrolysis by passing hot compressed water of a predetermined temperature (for example, about 150 to 300° C.) and a predetermined pressure or more (for example, saturated vapor pressure or more) in a feedstock supplied from the outside for a predetermined time and generating monosaccharides from these polysaccharides. Such a biomass treatment device A is used in generating monosaccharides which become the feedstock of alcohol fermentation in a plant for manufacturing bioethanol from a biomass (a biogenous resource excluding fossil resources), for example.

The present applicant proposed an apparatus and a method for treating a biomass in Japanese Patent Application No. 2009-219362 (filed on Sep. 24, 2009, title of invention: APPARATUS AND METHOD FOR TREATING BIOMASS), in which xylooligosaccharide and cellooligosaccharide are obtained individually from polysaccharides (carbohydrates) contained in a biomass (a wood-based biomass) by adjusting a hot-water temperature in a hot compressed water reaction device (a former-stage saccharification device); xylooligosaccharide is treated by a first catalytic reaction device (a latter-stage saccharification device) to monosaccharify it to xylose (C₅H₁₀O₅: pentose); cellooligosaccharide is treated by a second catalytic reaction device (a latter-stage saccharification device) to monosaccharify it to glucose (C₆H₁₂O₆: hexose); and further bioethanol (C₂H₆O) is manufactured by fermentation treatment of xylose with a first fermentation device, and by fermentation treatment of glucose with a second fermentation device.

As is well known, the main components of the wood-based biomass are cellulose (polysaccharide), hemicellulose (polysaccharide) and lignin. By passing hot water through the wood-based biomass of such components, it is possible to further decompose cellulose or hemicellulose into polysaccharides with a lower degree of polymerization (xylooligosaccharide, cellooligosaccharide and various oligosaccharides with the degree of polymerization slightly higher than these).

The hot compressed water reaction device 1 is a hot water flow-type reaction device which may hydrolyze a wood-based biomass under a first reaction condition for decomposing hemicellulose to generate a first polysaccharide solution containing the xylooligosaccharide, and then continue to hydrolyze the wood-based biomass with hot compressed water under a second reaction condition for decomposing cellulose to generate a second polysaccharide solution containing the cellooligosaccharide. Here, the hot compressed water is water in subcritical state, meaning hot water pressurized to maintain a liquid state.

Specifically, the hot compressed water reaction device 1 includes a pump 1a, first heat exchangers 1b and 1b′, a second heat exchanger 1c, a heater 1d, a water flow control valve 1e, a reaction vessel 1f, a brancher 1g, and a controller 1h.

The pump 1a pressurizes water supplied from the outside and delivers the pressurized water to each of the first heat exchangers 1b and 1b′.

The first heat exchanger 1b heats the compressed water introduced from the pump 1a by heat exchange with the first monosaccharide solution and delivers the heated compressed water to the second heat exchanger 1c as hot compressed water. Further, details of the first monosaccharide solution will be described later.

The first heat exchanger 1b′ heats the compressed water introduced from the pump 1a by heat exchange with the second monosaccharide solution and delivers the heated compressed water to the second heat exchanger 1c as hot compressed water. Further, details of the second monosaccharide solution will be described later.

The second heat exchanger 1c heats the hot compressed water introduced from the first heat exchangers 1b and 1b′ by heat exchange with the first and second polysaccharide solutions, and delivers the heated hot compressed water to the heater 1d as hot compressed water. Further, details of the first and second polysaccharide solutions will described later.

The heater 1d heats the hot compressed water introduced from the second heat exchanger 1c to the temperature at which hydrolysis of the wood-based biomass is possible, according to temperature control signals input from the controller 1h, and delivers this hot compressed water to the water flow control valve 1e.

The water flow control valve 1e is an electrical control valve whose degree of opening is regulated depending on flow rate control signals input from the controller 1h, and delivers the hot compressed water introduced from the heater 1d to the reaction vessel 1f after adjusting its water flow rate.

The reaction vessel 1f is a vessel, whose internal space is filled with a predetermined amount of the wood-based biomass supplied from the outside. It is configured so that the hot compressed water introduced from the water flow control valve 1e is delivered to the brancher 1g at the latter stage after the hot compressed water passes through the wood-based biomass. In addition, as the hot compressed water passes continuously through the reaction vessel 1f, the wood-based biomass is hydrolyzed. The hot compressed water is delivered to the brancher 1g as a polysaccharide solution containing polysaccharides generated by the hydrolysis of the wood-based biomass.

The brancher 1g selectively delivers the polysaccharide solution introduced from the reaction vessel 1f to either one of the first catalytic reaction device 2 and the second catalytic reaction device 3, depending on branching control signals input from the controller 1h. In addition, the polysaccharide solution delivered from the reaction vessel 1f is at a high temperature (150° C. to 270° C.), and thus it is preferable to introduce the polysaccharide solution into the brancher 1g after cooling the polysaccharide solution.

The controller 1h outputs temperature control signals to the heater 1d, and outputs flow rate control signals to the water flow control valve 1e to control the temperature and flow rate (the feed amount) of the hot compressed water to be delivered to the reaction vessel 1f. Thus, it has a function of selectively switching the first reaction condition for decomposing the hemicellulose and the second reaction condition for decomposing the cellulose. Here, the first reaction condition is a condition for decomposing the hemicellulose contained in the wood-based biomass and generating polysaccharides with xylooligosaccharide as a main component, and the second reaction condition is a condition for decomposing the cellulose contained in the wood-based biomass and generating polysaccharides with cellooligosaccharide as a main component. The first reaction condition and the second reaction condition are defined as a combination of the ratio K (=Q/V) of the feed amount Q (ml) of the hot compressed water and the feed amount V (g) of the wood-based biomass and the temperature T (° C.) of the hot compressed water.

In the reaction vessel 1f, the controller 1h controls the temperature T and the feed amount Q of the hot compressed water so that the wood-based biomass can be hydrolyzed first under the first reaction condition, and then controls the temperature T and the feed amount Q of the hot compressed water so that the wood-based biomass is hydrolyzed under the second reaction condition. In this way, the hot compressed water delivered from the reaction vessel 1f contains a polysaccharide solution (a first polysaccharide solution) with xylooligosaccharide as a main component under the first reaction condition, and the hot compressed water delivered from the reaction vessel 1f becomes a polysaccharide solution (a second polysaccharide solution) with cellooligosaccharide as a main component under the second reaction condition.

Furthermore, the controller 1h controls the brancher 1g so that the hot compressed water (the first polysaccharide solution) delivered from the reaction vessel 1f is delivered to the first catalytic reaction device 2 under the first reaction condition, and controls the brancher 1g so that the hot compressed water (the second polysaccharide solution) delivered from the reaction vessel 1f is delivered to the second catalytic reaction device 3 under the second reaction condition.

Using the solid-acid catalyst, the first catalytic reaction device 2 hydrolyzes the first polysaccharide solution, introduced from the hot compressed water reaction device 1 (strictly speaking, from the brancher 1g) under the first reaction condition, into the first monosaccharide solution containing xylose. In addition, the first catalytic reaction device 2 includes a first mixing device 2a and a first solid-liquid separation device 2b.

The first mixing device 2a facilitates a decomposition reaction (that is, a saccharification reaction) by agitating and mixing the first polysaccharide solution introduced from the hot compressed water reaction device 1 and the solid-acid catalyst filled in advance. By such a saccharification reaction, the xylooligosaccharide contained in the first polysaccharide solution is decomposed into xylose which is a monosaccharide. Then, a first liquid mixture containing the first monosaccharide solution containing xylose generated like this and the solid-acid catalyst is delivered from the first mixing device 2a to the first solid-liquid separation device 2b

The first solid-liquid separation device 2b separates the first liquid mixture introduced from the first mixing device 2a into the first monosaccharide solution containing xylose and the solid-acid catalyst by performing solid-liquid separation, and recovers the separated solid-acid catalyst, and supplies (reuse) the solid-acid catalyst to the first mixing device 2a. Then, the first monosaccharide solution containing xylose is delivered to the first fermentation device. As such a first solid-liquid separation device 2b, a settlement tank can be used. That is, in the first liquid mixture supplied to the settlement tank, the solid-acid catalyst, which is a solid, precipitates at the bottom of the tank, and a supernatant liquid is obtained as the first monosaccharide solution containing xylose. The first fermentation device generates ethanol by alcohol-fermenting the first monosaccharide solution.

Using the solid-acid catalys, the second catalytic reaction device 3 generates a second monosaccharide solution containing glucose by hydrolyzing the second polysaccharide solution introduced from the hot compressed water reaction device 1 (strictly soeaking, from the brancher 1g) under the second reaction condition. The second catalytic reaction device 3 includes a second mixing device 3a and a second solid-liquid separation device 3b.

The second mixing device 3a enhances hydrolysis (that is, a saccharification reaction) by agitating and mixing the second polysaccharide solution introduced from the hot compressed water reaction device 1 and the solid-acid catalyst filled in advance to make both contact. By such a saccharification reaction, the cellooligosaccharide contained in the second polysaccharide solution is decomposed into glucose, which is a monosaccharide. The second monosaccharide solution containing glucose produced like this and the second liquid mixture containing a solid-acid catalyst are delivered from the second mixing device 3a to the second solid-liquid separation device 3b.

The second solid-liquid separation device 3b separates the second liquid mixture introduced from the second mixing device 3a into the second monosaccharide solution containing glucose and the solid-acid catalyst by the solid-liquid separation, and recovers the separated solid-acid catalyst, and supplies (reuse) the solid-acid catalyst to the second mixing device 3a. Then, the second monosaccharide solution containing glucose to the second fermentation device. As such a second solid-liquid separation device 3b, a settlement tank can be used as in the first solid-liquid separation device 2b. That is, in the second liquid mixture supplied to the settlement tank, the solid-acid catalyst, which is a solid, precipitates at the bottom of the tank, and a supernatant liquid is obtained as the second monosaccharide solution containing glucose. The second fermentation device generates ethanol by alcohol-fermenting the second monosaccharide solution.

Next, a method of heating the hot compressed water in the biomass treatment device A configured as described above will be described.

First, half of compressed water delivered from the pump 1a is supplied to the first heat exchanger 1b, and the remaining half is supplied to the first heat exchanger 1b′. In each of the first heat exchangers 1b and 1b′, compressed water is heated by heat exchange with the first monosaccharide solution or the second monosaccharide solution. Thereafter, the hot compressed water delivered from the first heat exchangers 1b and 1b′ is joined to be introduced into the second heat exchanger 1c. The hot compressed water is further heated by heat exchange with the first polysaccharide solution or the second polysaccharide solution in the second heat exchanger 1c. The first polysaccharide solution and the second polysaccharide solution are at higher temperatures than the first monosaccharide solution and the second monosaccharide solution. Then, the hot compressed water is delivered to the heater 1d and heated in the heater 1d. At this time, the controller 1h controls the heater 1d to heat the hot compressed water with minimal required amount of energy to the temperature at which hydrolysis of the wood-based biomass can be hydrolyzed.

As mentioned above, in the present embodiment, the compressed water is first heated by heat exchange with the first monosaccharide solution in the first heat exchanger 1b and heat exchange with the second monosaccharide solution in the first heat exchanger 1b′. The hot compressed water delivered from the first heat exchangers 1b and 1b′ is further heated by heat exchange with the first polysaccharide solution or the second polysaccharide solution in the second heat exchanger 1c. Then, this hot compressed water is heated with minimal required amount of energy in the heater 1d to the temperature at which the wood-based biomass can be hydrolyzed. Thus, in the present embodiment, it is possible to heat the hot compressed water with a low amount of energy in the heater 1d to the temperature at which he wood-based biomass can be hydrolyzed, since the hot compressed water is heated by heat exchange in the first heat exchangers 1b and 1b′ and the second heat exchanger 1c. That is, in the present embodiment, it is possible to improve the energy efficiency in heating of the hot compressed water more than in the related art, since the hot compressed water is preheated using the heat in the first monosaccharide solution, the second monosaccharide solution, and the first polysaccharide solution and the second polysaccharide solution.

While the embodiment of the present invention has been described as above, the present invention is not limited to the embodiment. But, variations as in the following example may be conceived.

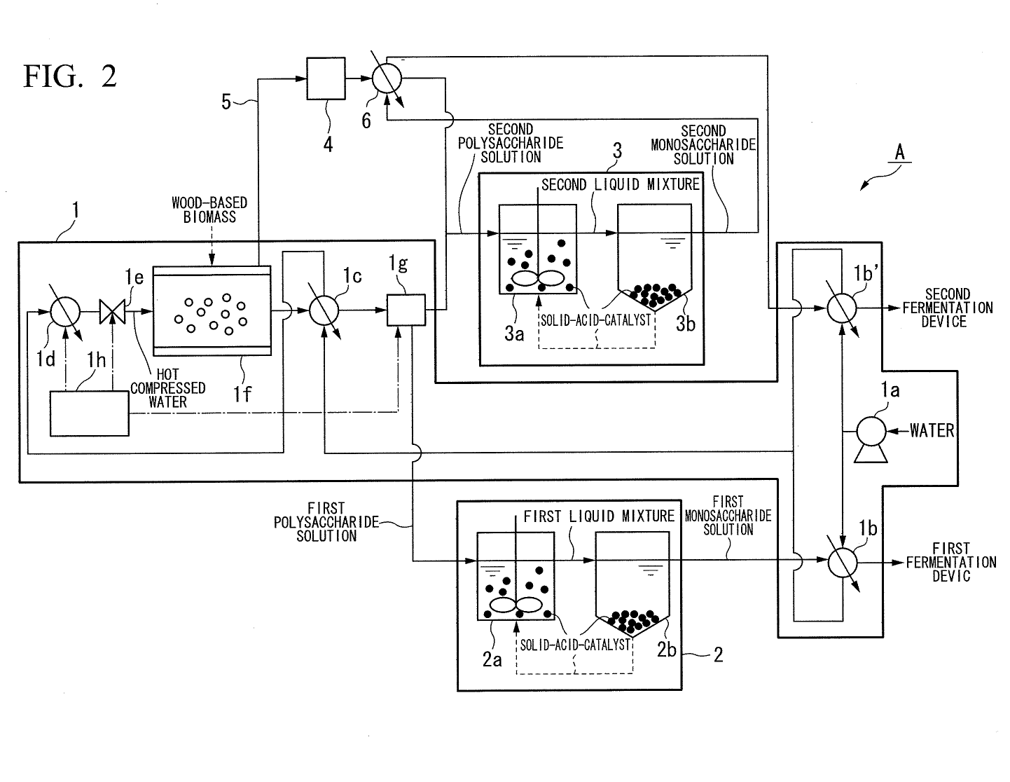

FIG. 2 shows a modified example of an embodiment of the present invention.

After reaction under the second reaction condition, solids including cellulose remain in the reaction vessel 1f. In this modified example, the solids including the cellulose, which remains in the reaction vessel 1f after reaction under the second reaction condition, are moved to the enzyme reactor 4 together with the hot compressed water, and are hydrolyzed by an enzyme (cellulase) in the enzyme reactor 4, and this is made into a polysaccharide solution with cellooligosaccharide as a main component. In FIG. 2, a flow channel 5 is formed from the reaction vessel 1f to the second catalytic reaction device 3, and the enzyme reactor 4 is installed on this flow channel 5. In this case, as described above, the hot compressed water delivered from the reaction vessel 1f is at a high temperature (150° C. to 270° C.). Thus, it is necessary to decompress the hot compressed water down to atmospheric pressure in advance at the time of inflow into the enzyme reactor 4 and cool the hot compressed water to a temperature (50° C. or below) which is optimal for the enzyme reaction.

Meanwhile, the optimal temperature for hydrolysis of the polysaccharide solution in the second catalytic reaction device 3 is about 100° C. Therefore, it is preferable to have the temperature of polysaccharide solution rise to about 100° C. in advance at the time when the polysaccharide solution obtained by decomposition of cellulose in the enzyme reactor 4 inflows into the second catalytic reaction device 3.

In this modified example, a third heat exchanger 6 is installed on the flow channel 5 between the enzyme reactor 4 and the second catalytic reaction device 3. Heat exchange is performed with the second monosaccharide solution delivered from the second catalytic reaction device 3 with the third heat exchanger 6, so that the polysaccharide solution leading from the enzyme reactor 4 to the second catalytic reaction device 3 is heated.

In addition, the heating of the polysaccharide solution in the third heat exchanger 6 may be performed by heat exchange with the hot compressed water delivered from the reaction vessel 1f. Further, in FIG. 2, as the second monosaccharide solution used in heat exchange in the third heat exchanger 6, a monosaccharide solution between the second catalytic reaction device 3 and the first heat exchanger 1b′ is used. The monosaccharide solution between the heat exchanger 1b′ and the second fermentation device may be used as well.

Furthermore, variations as shown below can also be considered in the present invention.

In the above-mentioned embodiment, each half of the compressed water is heated in each of the first heat exchangers 1b and 1b′, and then unites, and then the hot compressed water that is joined thereafter is heated by the second heat exchanger 1c. But, the present invention is not limited thereto.

For instance, with either of the first heat exchanger 1b and the first heat exchanger 1b′ provided, the hot compressed water may be heated by the first heat exchanger 1b or the first heat exchanger 1b′. Also, either of the first heat exchangers 1b and 1b′ and the second heat exchanger 1c may be installed as well. In other words, although three heat exchangers were installed in the above-mentioned embodiment, it is also possible to provide at least one of the three heat exchangers in the invention.

### INDUSTRIAL APPLICABILITY

As described above, according to the present invention, it is possible to improve energy efficiency more than the related art in the saccharification treatment of the biomass.

### DESCRIPTION OF REFERENCE NUMERALS

A: biomass treatment device
1: hot compressed water reaction device
2: first catalytic reaction device
3: second catalytic reaction device
1a: pump
1b, 1b′: first heat exchanger
1c: second heat exchanger
1d: heater
1e: water flow control valve
1f: reaction vessel
1g: brancher
1h: controller
2a: first mixing device
2b: first solid-liquid separation device
3a: second mixing device
3b: second solid-liquid separation device
4: enzyme reactor
6: third heat exchanger

1. A biomass treatment device including a hot compressed water reaction device that hydrolyzes a biomass by passing hot compressed water to the biomass to prepare polysaccharides, and a solid-acid catalytic reaction device that generates monosaccharides from the polysaccharides using a solid-acid catalyst, the device comprising:
at least one of a first heat exchanger for heating the hot compressed water by the heat of a monosaccharide solution including the monosaccharides delivered from the solid-acid-catalytic reaction device and a second heat exchanger for heating the hot compressed water by the heat of a polysaccharide solution including the polysaccharides introduced into the solid-acid-catalytic reaction device from the hot compressed water reaction device.

2. The biomass treatment device according to claim 1, wherein, when the temperature of the monosaccharide solution is lower than that of the polysaccharide solution, the hot compressed water is heated by the second heat exchanger after the first heat exchanger.

3. The biomass treatment device according to claim 1, comprising a plurality of solid-acid-catalytic reaction devices,
wherein the hot compressed water reaction device delivers the polysaccharide solution to the solid-acid-catalytic reaction devices that are different according to the type of polysaccharides, and
wherein water for the hot compressed water is branched into multi-streams and each water stream is heated by the first heat exchanger that is installed in each of the solid-acid-catalytic reaction devices.

4. The biomass treatment device according to claim 1, comprising an enzyme reactor for hydrolyzing the remained biomass after the hot compressed water has passed with an enzyme.

5. The biomass treatment device according to claim 4, comprising a third heat exchanger for heating a solution delivered from the enzyme reactor,
wherein the solution heated by the third heat exchanger is delivered to the solid-acid-catalytic reaction device.

6. The biomass treatment device according to claim 5, wherein the solution delivered from the enzyme reactor is heated by the heat of the monosaccharide solution delivered from the solid-acid-catalytic reaction device in the third heat exchanger.
